(19) United States
(12) Patent Application Publication
(10) Pub. No.: US 2018/0334669 A1
(43) Pub. Date: Nov. 22, 2018

(54) Fault Tolerant Method of Tagging Pharmaceutical and Nutritional Products using Multiple Taggants in Varying Ratios

(71) Applicants: David R. Hall, Provo, UT (US); Steven J.M. Butala, Provo, UT (US); Dan Allen, Springville, UT (US); Andrew Nguyen, Provo, UT (US); Conrad Rosenbrock, Provo, UT (US); Ben Swenson, Lehi, UT (US); Daniel Hendricks, Provo, UT (US); Joe Fox, Spanish Fork, UT (US)

(72) Inventors: David R. Hall, Provo, UT (US); Steven J.M. Butala, Provo, UT (US); Dan Allen, Springville, UT (US); Andrew Nguyen, Provo, UT (US); Conrad Rosenbrock, Provo, UT (US); Ben Swenson, Lehi, UT (US); Daniel Hendricks, Provo, UT (US); Joe Fox, Spanish Fork, UT (US)

(21) Appl. No.: 15/597,633

(22) Filed: May 17, 2017

(51) Int. Cl. C12N 15/10

(52) U.S. Cl. CPC C12N 15/1065

(57) ABSTRACT

We disclose a method of tagging a variety of pharmaceutical or nutritional products in which well-studied chemicals may be added to the products in unique ratios. The identities of the chemicals and their relative ratios comprise unique taggants. The taggants may identify which of multiple distinct categories the product falls within. The method includes the step of systematically varying the relative concentrations of the chemicals resulting in multiple ratios of the chemicals. The plurality of ratios of a defined set of chemicals may be collected to form a library of taggants associated with specified items or categories. As the number of chemicals added per product increases, the library supports more categories and the system is less likely to produce a false positive. The method may result in a series of taggants for each item which is single fault tolerant or double fault tolerant.

### BACKGROUND

### Field of the Invention

This disclosure relates to devices and methods of tagging pharmaceutical and nutritional products, to identify and distinguish the item and for analyzing biological samples to identify consumed items.

### Background of the Invention

Detecting and verifying consumption of pharmaceutical or nutritional products, is a challenge for managing product distribution, patient treatment, drug abuse, counterfeit products, and tracking the sources of foods and food ingredients. Some active ingredients in pharmaceutical or nutritional products are delivered in quantities as small as 10-20 mg or even smaller. These small quantities make direct detection of the active ingredient challenging. Furthermore, many pharmaceutical or nutritional products are metabolized or partially metabolized in the body. Consequently, the concentration of the active ingredient and the concentration of metabolic by-products of the active ingredient in biological fluids and tissues varies over time.

Several methods have been used to identify consumed pharmaceutical or nutritional products in bodily waste. These methods include micro-scale consumable bar codes which pass through the digestive tract, unique surface markings, and unique combinations of pill shapes and colors. Detection of these unique features urine is not possible in some cases.

Chemical taggants have been used to a limited degree. This taggants are limited because components of pills must be sufficiently biologically inert that they do not adversely affect the delivery or activity of the active ingredients. A taggant optimized for properties such as optical or chemical detectability may not meet these requirements.

A series of taggants that may be added to pharmaceutical or nutritional products and which have a known safety profile, are biologically inert, and which are detectable in a biological sample is needed. Ideally, the series of taggants includes a large enough number to identify many data points along the chain of distribution.

### BRIEF SUMMARY OF THE INVENTION

We disclose a method of tagging pharmaceutical or nutritional products using chemicals which may not be unique to the product. The taggant may include multiple chemicals in defined ratios. The chemicals may be those which are often found in food or pharmaceutical or nutritional products. However, the disclosed method drastically reduces the likelihood of erroneous data points caused by extraneous chemicals. This is at least because the disclosed method includes the use of multiple chemicals, which may be at least four, eight, twenty, or more, in unique relative ratios. The identities of the chemicals and their ratios may function in a manner that is similar to bar codes. The more ratios of chemicals in the taggant, the more unlikely that an erroneous reading of a single chemical or ratio will be viewed as a valid data point.

The identity of the chemicals and their ratios may be collected into libraries. The libraries may include multiple categories of different types of information. The categories may include one or more of a manufacturing site, a distributor, a dispensing pharmacy, a retailer, a prescribing healthcare provider, a formulation, a drug, and a prescribed end-user. Libraries may be used separately or combined to provide more information about a product and to reduce the likelihood of an erroneous data point.

The chemicals used in the taggants may include food dyes, artificial sweeteners, nucleic acids, other natural or synthetic polymers, or combinations thereof. The chemicals may be detected using light absorption spectroscopy techniques which may be performed by a medical toilet.

### BRIEF DESCRIPTION OF THE DRAWINGS

FIG. 1 is a table illustrating the use of three chemicals to create taggants for three categories according to an embodiment of the disclosure.

FIG. 2 is a table illustrating the use of three chemicals to create taggants according to an embodiment of the disclosure in which one chemical is absorbed into a user's bloodstream faster than the other two chemicals.

FIG. 3 is a table illustrating the use of four chemicals to create taggants for six categories according to an embodiment of the disclosure.

FIG. 4 is a table illustrating the use of the disclosed system for identifying members of drug families.

FIG. 5 is a table illustrating a library of taggants according to the disclosure.

FIG. 6 is a table illustrating another library of taggants according to the disclosure which may be used alone or with the library of FIG. 5.

FIG. 7 is a flow chart that provides steps which may be performed in an embodiment of the disclosed method.

### DETAILED DESCRIPTION OF THE INVENTION

### Definitions

Drug, as used herein, means any pharmacologically or physiologically active agent or mixture of agents. Drug may also include an active ingredient in a health product, including a nutritional supplement. Drug may include one or more placebos.

Biological sample, as used herein, means urine, feces, whole blood, serum, plasma, cerebrospinal fluid, ascites, mucous, gastric gavage, saliva, breath, breast milk, or any combination thereof.

User, as used herein, means a patient, a participant in a medical study, or any individual who has consumed a drug composition which includes at least one taggant as described herein. The user may be animal or human.

Medical toilet, as used herein, means a device that may be used to collect one or more biological sample from a user. This may include a traditional water toilet. However, medical toilet, as used herein, may mean any device which may be used to collect biological samples according to the present disclosure and which may be equipped to analyze biological samples.

Consume, as used herein, means used in or on a human or animal body. This includes, but in not limited to, oral ingestion, topical or ophthalmic application, intravenous uptake, intraarterial uptake, subcutaneous uptake, sublingual uptake, and through a nebulizer mist. Consumable, means an item which may be consumed as defined herein.

Source of a main ingredient, as used herein, means the original plant, animal, or synthesized product that becomes the main active ingredient in a product or from which the main ingredient is isolated. This may include, but is not limited to, livestock, plants, live cultures, or synthesized pharmaceutical compounds.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

We disclose a method of manufacturing pharmaceutical or nutritional supplement products, where the ratios of known chemicals may be varied to create a unique taggant composition to identify each product or product category. Consequently, the “taggant” according to the disclosure, comprises both the multiple chemicals added to the products and their relative ratios.

Previous tagging methods have depended on unique or difficult to mimic tags. In contrast, the disclosed method uses chemicals which may be already used in pharmaceutical and nutritional supplement products or which may be present in foods, but which are used in a unique way according to the disclosure. Also, in contrast to the common method of using a single chemical as a taggant, we disclose a method in which several chemicals may be added to a product, which may be a pill, gel capsule, powder, liquid medicine, food ingredient, food source, or prepared food product. Each chemical may be an inert additive which may be present in other pharmaceutical products, nutritional supplement products, or foods. Accordingly, use of a single such chemical would provide a significant probability of false positives. However, as the number of chemicals used in the taggant increases as disclosed herein, additional ratios and additional measurement dimensions are added. In some embodiments, the taggants may include between three and ten chemicals. In some embodiments, the taggants include three chemicals. In some embodiments, the taggants include four chemicals. In some embodiments, the taggants include five chemicals. In some embodiments, the taggants include six, seven, eight, nine, or ten chemicals.

The number of chemicals determines the number of ratios of the chemicals that are possible. For a number “n” of unique chemicals, the number of ratios that include two of the chemicals is “n−1.” In some embodiments, the number of ratios may be between three and nine. In some embodiments, the number of ratios may be three. In some embodiments, the number of ratios may be four. In some embodiments, the number of ratios may be five. In some embodiments, the number of ratios may be six. In some embodiments, the number of ratios may be seven. In some embodiments, the number of ratios may be eight. In some embodiments, the number of ratios may be nine.

By over-constraining the design, in other words, by using more taggant dimensions (degrees of freedom) than are strictly necessary to uniquely identify the product or category, it becomes possible to discriminate false positives. This is the case because the chemical array concentrations may be designed such that there is a low likelihood that the random presence of a single chemical from another source will result in a valid reading. This results in a method that is single fault tolerant or even double fault tolerant.

For example, despite a single error, the following sequence or bar code may be readily identified: [0,1,2,3,4,11,6,7,8,9]. It is a list of numbers from 0 to 9, where 5 has been replaced by the incorrect value 11. The overlap or dot-product of this vector with the correct vector [0,1,2,3,4,5,6,6,7,9] is large, and by removing a single positive errant component, the overlap becomes perfect. This method takes advantage of the knowledge that any error is most likely positive. In other words, one must primarily consider the possibility that a concentration of a detected chemical has increased due to co-consumption of a product containing the taggant.

The chemicals that may be used in taggants according to the disclosure need not be unique to the pharmaceutical or nutritional product. In some embodiments, the chemicals may include food dyes, artificial sweeteners, nucleic acids, other natural or synthetic polymers, or combinations thereof. In some embodiments, the chemicals may include one or more of polyethylene glycol, ethylene-vinyl acetate, copovidone, povidone, propylparaben, methyl paraben, acesulfame potassium, mannitol, sorbitol, xylitol, steviol glucuronide, sucralose, oleic acid, trans-anethole, 1, 8-eucalyptol, limonene-2D, linalool, citronellol, riboflavin, tartaric acid, salts of tartaric acid, deoxyribonucleic acid, ribonucleic acid, nucleic acid analogs, or combinations thereof.

Another detail of the disclosure which reduces the likelihood of error, is that the disclosed method may include a library of taggants which functions similar to unique fingerprints. The libraries may include unique ratios of chemicals which may be added to each product and may be designed such that a single positive error is unlikely to be interpreted to be a valid library entry.

The disclosed method relies on the lower likelihood of coincident probabilities to improve accurate detection statistics. The logic may be compared to techniques used for robust signal coding, which include grey codes which require two bit flips to change number. The disclosed method uses multiple chemicals at varying ratios, rather than bar code numerals, which decrease the likelihood of an erroneous precise overlap. The relative ratios may still be unique in the presence of a single erroneously determined ratio. In some embodiments, the likelihood of coincident probabilities is reduced by providing a number of available detectably distinct ratios which is greater than a number of categories of information. In an example, the categories may include one or more of a manufacturing site, a distributor, a dispensing pharmacy, a retailer, a prescribing healthcare provider, a formulation, a drug, and a prescribed end-user. A category may have a single entry, the product itself. Therefore, the method may distinguish each of the pharmaceutical or nutritional products. Also in an example, the product categories may be pills and the mass shared by the taggants may be about 100 mg. In another example the product categories may be gel capsules, the mass shared by the taggants may be about 100 mg, and the taggants may be liquids. In a further example, the product categories may be liquids or syrups. With typical dosage volumes of about 5-15 ml, the taggant volumes may be 100 ul or more up to milliliter quantities for very water-soluble chemicals used in the taggants. In another example, the nutritional product may include milk, wheat, rice, sugar, salt, fruits, vegetables, olives, peanuts, cacao, coffee, tobacco, alcohols, beef, pork, chicken, poultry, fowl, fish, whales, mollusks, crustacea, seaweed, oils, or combinations thereof.

In some embodiments, the method may be used to identify a source of a main ingredient in a pharmaceutical or nutritional product. In relation to food products, the source of a main ingredient in a food product may indicate the source of the livestock in a meat product, the source of wheat in a baked good, or the source of a plant in a prepared vegetable dish or nutritional supplement.

In one embodiment of the disclosure, multiple urine samples are collected over time for the purpose of identifying a product category that is associated with a drug product the user has consumed. In this embodiment, the drug product includes a taggant according to the instant disclosure. The taggant may include multiple chemicals in defined ratios. After the user consumes the drug product, multiple urine samples are collected from the user at discrete intervals. The urine samples may represent a series of separate urination events. The trajectory of the relative concentrations of the chemicals in the taggant may be measured in each of the multiple urine samples. The measurements of the chemicals in the urine samples may be interpolated to identify the product category. In other words, a single observation may not be conclusive, but a second observation may constrain the classification process.

The chemicals in the taggants may be detected using a variety of techniques, including light absorption spectroscopy. In some embodiments, the light absorption spectroscopy may include one or more of near infrared, visible, and ultraviolet spectroscopy.

In some embodiments, the light absorption spectroscopy may be conducted using a medical toilet. The user may deposit a biological sample into a bowl of the medical toilet. The biological sample may include urine, feces, whole blood, serum, plasma, cerebrospinal fluid, ascites, mucous, gastric gavage, saliva, breath, breast milk, or any combination thereof. The medical toilet may analyze the biological sample using light absorption spectroscopy and the measurements transmitted to a controller for data analysis.

In some situations, a nonlinear or time-dependent classification process may be suited to identify one or more categories associated with a consumed pharmaceutical or nutritional product. For transient consumption events where concentrations from previous ingestions are negligible, the trajectory may be distinct such that a product category may be determined. Alternatively, in situations in which a user consumes a drug product regularly, quasi-equilibrium or diurnal cyclic ratios may be used to categorize both drug identity and validate or confirm time and date of consumption.

In some embodiments, the number of categories may be between four and eight. In some embodiments, the number of categories may be four. In some embodiments, the number of categories may be five. In some embodiments, the number of categories may be six. In some embodiments, the number of categories may be seven. In some embodiments, the number of categories may be eight.

An aspect of the disclosed method is using multiple chemicals in systematically varied ratios as taggants to create detectably distinct libraries of taggants. In one embodiment, an excess of taggant ratio states relative to the number of product categories is provided. Due at least to the presence of the excess taggant ratios, the library is at least partially single fault tolerant, or redundant.

Another aspect of the invention is the use of multiple compounds whose ratios are detectably distinct. In an example, samples of a user's urine are collected, each from multiple urination events. Each urine sample is maintained separately from the others and analyzed as a separate sample as opposed to mixed into one solution. In other words, the multiple urine samples are not combined prior to analysis. In some examples, the multiple urination events may occur hours apart. The urine samples may be independently analyzed and the chemicals used in the taggant may be measured to estimate the relative ratios of the chemicals. The data may then be sent to a time-dependent model for categorization or classification of the consumed product and/or estimation of the consumption time or quantity. In embodiments in which the ratios of chemicals present signals correlated to concentration, the classification may be trained on calibrated data without resorting to calculation of explicit ratios.

The systematic variation of a ratio or concentration of inactive drug ingredients to create detectably distinct product categories opens unique opportunities for drug identification and tracking, without the need to detect the drug itself and also provides some robustness to potential false positives. Large libraries have the ability to code for many product categories and optionally information as detailed as the manufacturer, distributor, prescriber, pharmacy, formulation, drug, or prescribed user.

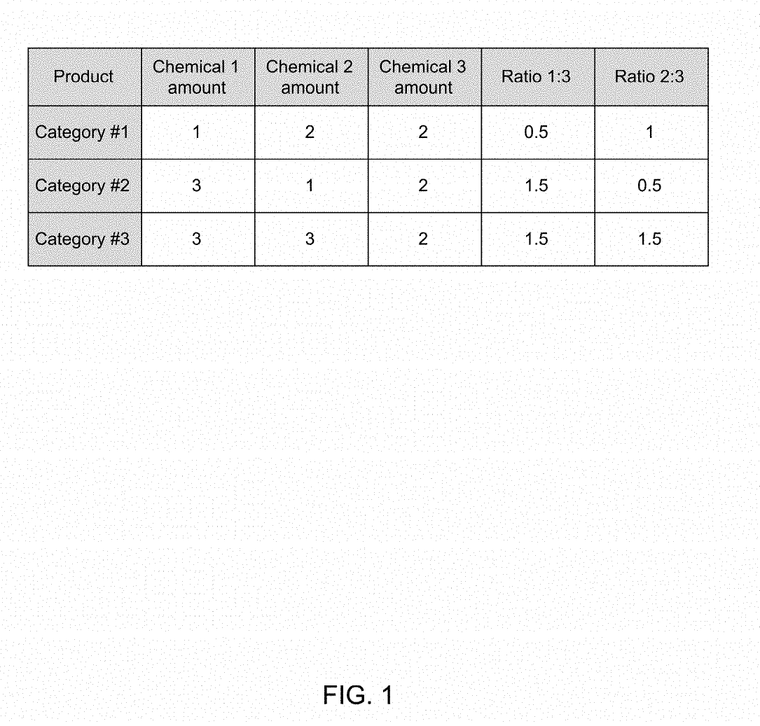

Referring now to the Figures, FIG. 1 provides a table which is an example of the disclosed tagging system comprising three distinct chemicals. The chemicals are referred to as chemical 1, chemical 2, and chemical 3. In this example, there are three categories of product referred to as category 1, category 2, and category 3. The table of FIG. 1 references two members of each of the three categories. Each member is associated with a different ratio of the three chemicals. Specifically, the ratios include either the ratio of chemical 1: chemical 3 or the ratio of chemical 2: chemical 3. Each of the two members of each of the three categories is represented by a different ratio of either chemical 1: chemical 3 or of chemical 2: chemical 3. A biological sample collected from a user who has consumed a product from one of the three categories may be analyzed. The category of the product, and possibly, the member of the category may be identified by determining the ratios of chemical 1: chemical 3 or of chemical 2: chemical 3 present in the biological sample. Consequently, the absolute amounts of the chemicals are not required to identify the category or the member of the category.

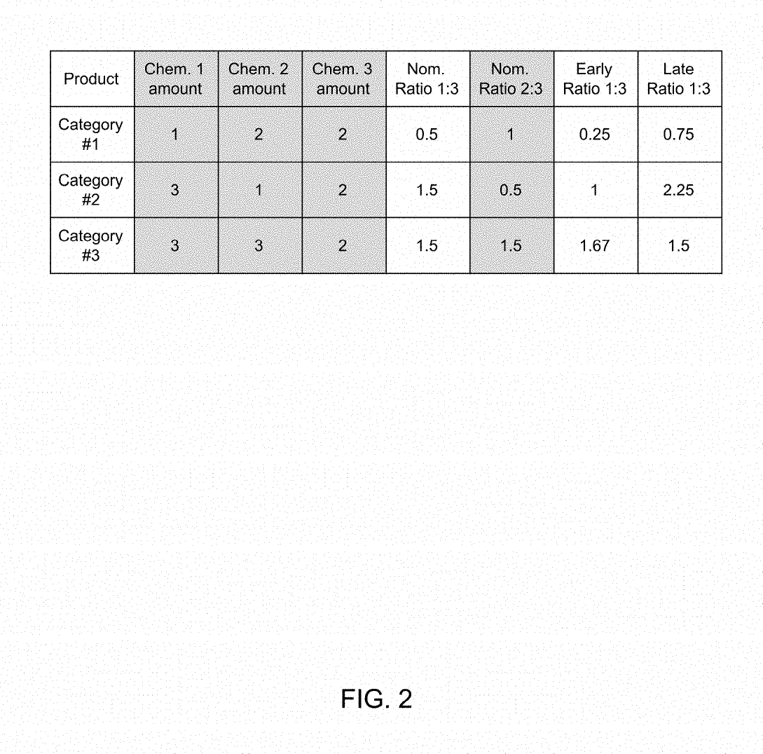

FIG. 2 illustrates an example in which the three chemicals of Figure enter the bloodstream at rates when consumed. Specifically, chemical 1 enters the bloodstream faster than chemical 2 and chemical 3 while all three chemicals have similar glomerular filtration rates. Metabolic processing of each chemical is negligible. User's consume the products labeled with the chemicals according to the table in FIG. 2. Then multiple urine samples are collected from the users. Each urine sample is analyzed to detect the three chemicals.

Because the users' bodies process chemical 1 at a different rate that chemicals 2 and 3, the ratios of chemical 1: chemical 3 vary over time, thus changing from the nominal ratio (referred to as Nom. Ratio in the table), while the ratios of chemical 2: chemical 3 remain relatively steady. In theory, the analysis of the data would include steps to consider variations in the ratios of chemical 1: chemical 3 over time. However, the bladder acts as a long duration integrator which may have a time constant of approximately 1-3 hours, which is on the order of typical uptake rates. Accordingly, the error from variation of the ratios of chemical 1: chemical 3 may be small. Furthermore, the evolution of the ratios of chemical 1: chemical 3 and chemical 2: chemical 3 may define a path through a 2-dimensional space which additionally reduces the likelihood of misinterpretation of the data.

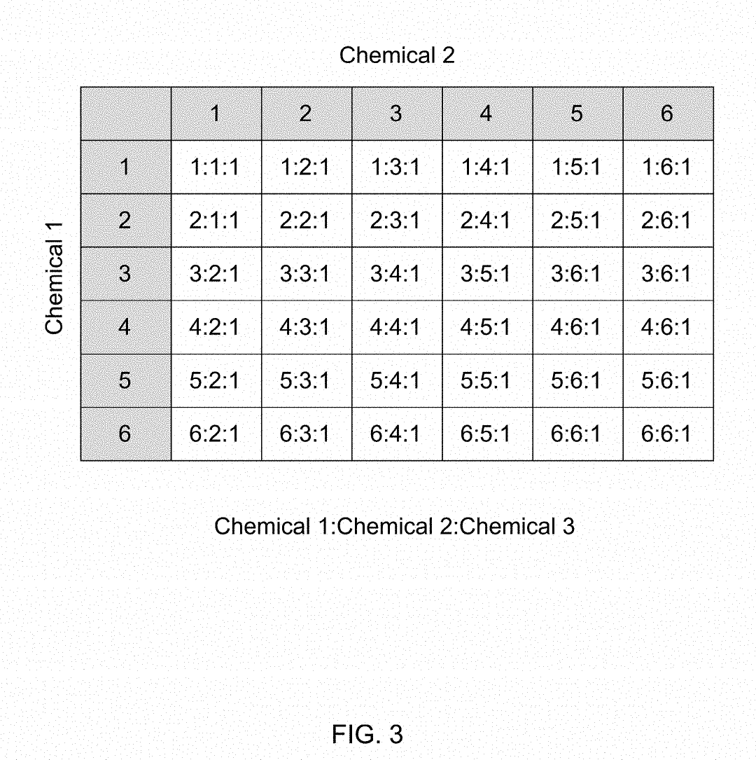

FIG. 3 illustrates an example in which the three chemicals of FIGS. 1 and 2 are all three used in systemically varied ratios to differentiate the three categories. This example illustrates the additional power that accompanies the use additional ratios in the taggant associated with each category or each member of a category. In this example, one valid ratio of the three chemicals uniquely identifies a product. Two chemical concentrations must both be altered, and in a precise way, to create a valid false positive. The more chemicals included in the taggants, the more concentrations of chemicals must be altered, for example, by detecting background chemical or erroneous measurements, to mimic a valid taggant.

Note that total amounts of chemicals do not necessarily distinguish product categories. This is relevant at least because a measurement of amount of chemicals in a biological sample may be uncertain due to variations in kinetics of events including uptake and elimination of the chemicals, and other variables that impact the biological sample. For example, the concentration of the chemicals detected in a urine stream may vary with urine output, uptake of the chemicals, glomerular filtration rate of the chemicals, and time of consumption of the product.

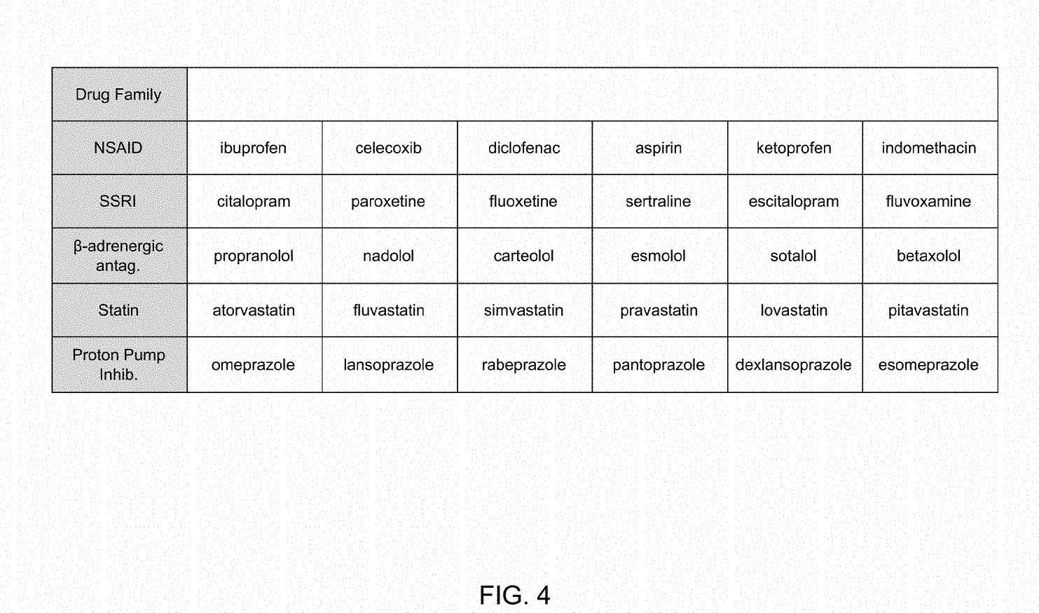

FIG. 4 provides a table that includes five families of compounds which may be active ingredients in pharmaceutical products. The families include nonsteroidal anti-inflammatory drug (NSAID), serotonin reuptake inhibitor (SSRI), β adrenergic receptor antagonist, and statin. Each family includes six different members as shown in FIG. 4. Unique ratios of a set of chemicals may define not only the drug family but also the member of the drug family.

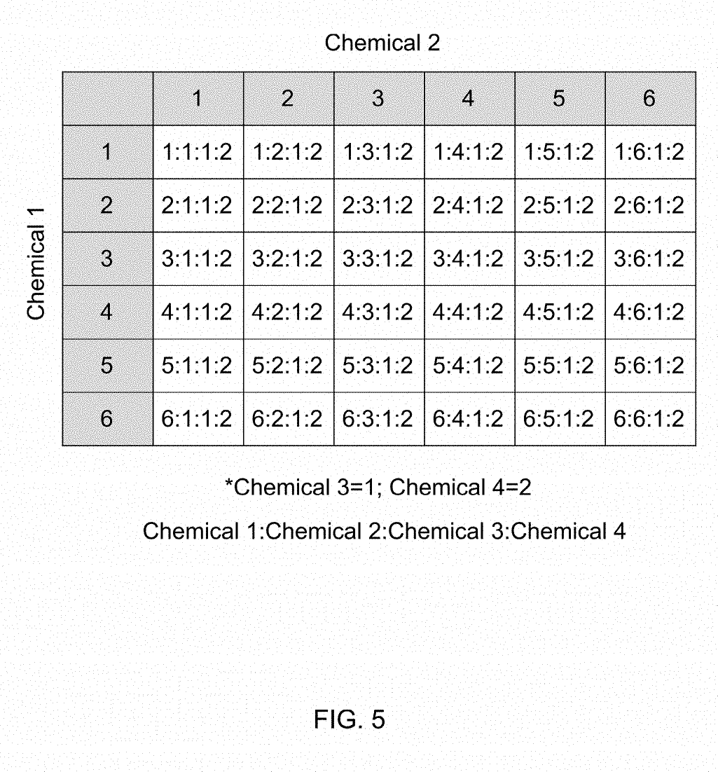

FIG. 5 includes a table which shows an example of a library which represents a series of six categories. The categories may be, for example, different drug families produced by the same manufacturer. Four distinct chemicals, chemical 1, chemical 2, chemical 3, and chemical 4, are used in varying ratios. In this embodiment, the relative proportions of chemical 3 and chemical 4 are maintained constant. Specifically, chemical 3 is 1 in each ratio and chemical 4 is 2 in each ratio. These two constant variables may, at least in part, define the manufacture. The addition of the ratios of chemicals 1 and 2 provide information about the drug families (for example, NSAID), each representing a category, and the members of the drug family, each representing a member. Each member of the category may be a specific active ingredient in a drug product (for example, ibuprofen).

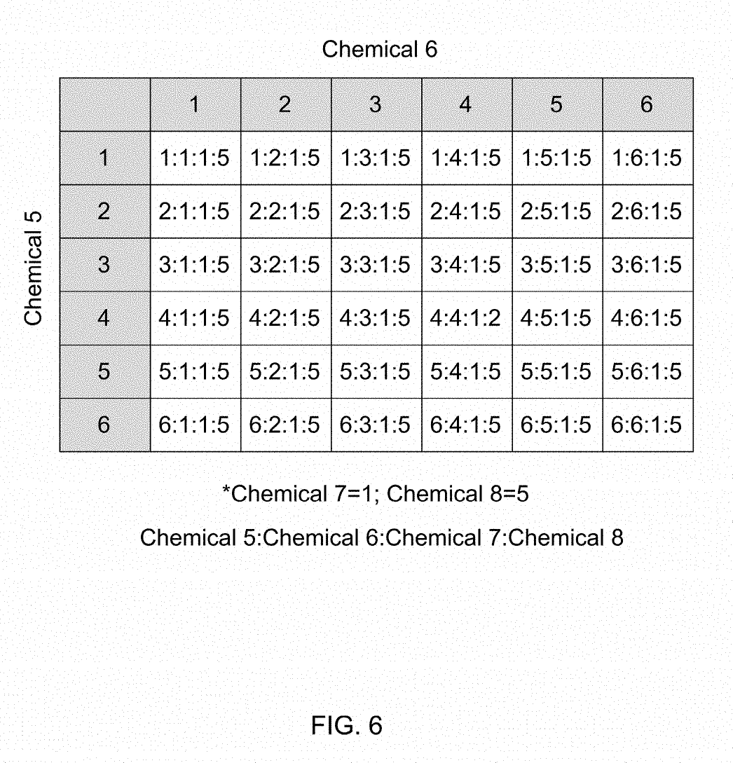

FIG. 6 includes a table which shows an example of an additional library, which may be used alone, or in combination with the library of FIG. 5. The table in FIG. 6 includes four chemicals which are detectably unique relative to those of FIG. 5. These chemicals are referred to as chemical 5, chemical 6, chemical 7, and chemical 8. Similar to the library of FIG. 5, the relative amounts of chemicals 7 and 8 are maintained constant at 1 and 5 respectively. The taggants in the library of FIG. 6 may provide information about drug or nutritional products which is of a different type than that provided by the library illustrated in FIG. 5. For example, the library of FIG. 6 may be used by a drug distributor (wholesaler) which distributes drug products to a variety of pharmaceutical retailers. The six categories may each represent a different pharmaceutical retailer, for example six different pharmaceutical retail chains. The members within each category may represent specific pharmacy within a retail chain. When used together, the libraries of FIGS. 5 and 6 may provide the equivalent of an elaborate “bar code” made of chemical ratios. The eight chemicals represented in the two libraries used together reduce the likelihood of a false positive relative to the likelihood associated with either library alone.

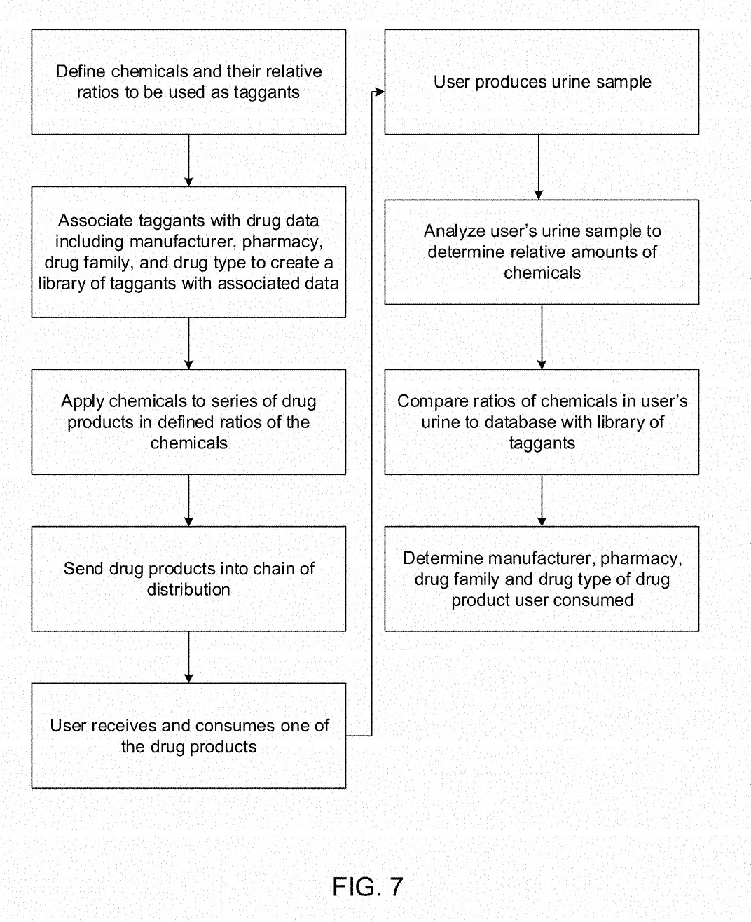

FIG. 7 includes a flow chart which illustrates steps which may be taken when using an embodiment of the disclosed method. First, the chemicals to be used in the taggants and their relative ratios are chosen. Each group of chemicals in a defined ratio represents a unique taggant. Next the unique taggants are associated with multiple types of information relating to pharmaceutical products. This information may relate to the manufacturer, pharmacy, drug family, and drug type. The unique taggants are associated in libraries which comprise different categories of each type of information. Next, the chemicals are applied to various drug products in the defined ratios and the tagged drug products are sent into the chain of distribution.

Eventually, a user obtains and consumes one or more of the tagged drug products. The user may provide a urine sample which may be analyzed to detect the relative concentrations of the chemicals in the taggants. The relative ratios of the chemicals in the user's urine are compared to the library of taggants to identify the information about the consumed drug product including the manufacturer, pharmacy, drug family and drug type of drug product.

While specific embodiments have been described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

1. A method of tagging pharmaceutical or nutritional products for later identification comprising the steps of:
a. providing a plurality of pharmaceutical or nutritional products;
b. assigning each of the plurality of pharmaceutical or nutritional products to one or more of between four and eight categories;
c. providing between three and ten spectrally distinct chemicals comprising food dyes, artificial sweeteners, synthetic polymers, or combinations thereof;
d. adding a unique combination of the between three and ten spectrally distinct chemicals to each of the plurality of pharmaceutical or nutritional products in between three and nine spectrally distinct ratios of chemical amounts systemically varying the relative concentrations of the between three and ten spectrally distinct chemicals to create a plurality of tagged products,
e. associating the identity of the between three and ten spectrally distinct chemicals and the between three and nine spectrally distinct ratios with the between four and eight categories; and
f. analyzing a biological sample collected from a user who has consumed at least one of the plurality of tagged products using a medical toilet, the medical toilet comprising a traditional water toilet, the traditional water toilet comprising a light absorption spectrometer.

2. The method of claim 1, further comprising the step of associating at least one of the unique combination of the between three and ten spectrally distinct chemicals with at least one of the plurality of pharmaceutical or nutritional products.

3. The method of claim 1, wherein a number of ratios within the between three and nine spectrally distinct ratios of chemical amounts is greater than a number of categories within the between four and eight categories.

4. (canceled)

5. The method of claim 1, wherein the plurality of pharmaceutical or nutritional products comprises drug products.

6. The method of claim 1, wherein the between four and eight categories consists of one or more of the following categories selected from the group consisting of: a manufacturing site, a distributor, a dispensing pharmacy, a retailer, a prescribing healthcare provider, a formulation, a pharmaceutical compound, a type of nutritional supplement, and a prescribed end-user.

7. The method of claim 1, wherein the plurality of pharmaceutical or nutritional products comprises nutritional products.

8. The method of claim 1, wherein the between four and eight categories consists of one or more of the following categories selected from the group consisting of: a manufacturing site, a distributor, a formulation, an intended use, and a source of a main ingredient.

9. The method of claim 1, wherein the between three and ten spectrally distinct chemicals comprises compounds which are detectable in urine by light absorption spectroscopy.

10. The method of claim 9, wherein the light absorption spectroscopy technique is selected from the group consisting of: near infrared spectroscopy, ultraviolet-visible spectroscopy, or both near infrared spectroscopy, and ultraviolet-visible spectroscopy.

11. The method of claim 1, wherein systematically varying relative concentrations of the between three and ten spectrally distinct chemicals comprises incrementally varying the concentration of a first spectrally distinct chemical relative to a second spectrally distinct chemical.

12. The method of claim 1, wherein the between three and nine spectrally distinct ratios of chemical amounts is unique in the presence of a single erroneously determined ratio.

13. The method of claim 1, wherein the between three and ten spectrally distinct chemicals comprises at least eight spectrally distinct chemicals.

14. (canceled)

15. The method of claim 1, wherein the between three and ten spectrally distinct chemicals comprises a food dye.

16. The method of claim 1, wherein the between three and ten spectrally distinct chemicals comprises an artificial sweetener.

17. The method of claim 1, wherein the between three and ten spectrally distinct chemicals comprise a chemical which is detectable by a near infrared spectroscopy, wherein the chemical is independently selected from the group consisting of: polyethylene glycol, ethylene-vinyl acetate, copovidone, povidone, propylparaben, methyl paraben, acesulfame potassium, mannitol, sorbitol, xylitol, steviol glucuronide, and sucralose, oleic acid, trans-anethole, 1, 8-eucalyptol, limonene-2D, linalool, citronellol, riboflavin, tartaric acid, and salts of tartaric acid.

18. The method of claim 1, further comprising the step of grouping unique taggants which are assigned to related items into a library.

19. The method of claim 1, wherein the plurality of pharmaceutical or nutritional products comprises non-ingestible products.

20. The method of claim 1, wherein each of the plurality of pharmaceutical or nutritional products is independently selected from the group of processed foods consisting of: milk, wheat, rice, sugar, salt, fruits, vegetables, olives, peanuts, cacao, coffee, tobacco, alcohols, beef, pork, chicken, poultry, fowl, fish, whales, mollusks, crustacea, seaweed, and oils.
